(19) United States
(12) Patent Application Publication
(10) Pub. No.: US 2015/0033832 A1
(43) Pub. Date: Feb. 5, 2015

(54) APPARATUS FOR REMOTELY MEASURING OUTDOOR WATER QUALITY AND METHOD THEREOF

(71) Applicant: KOREA INSTITUTE OF GEOSCIENCE AND MINERAL RESOURCES, Daejeon (KR)

(72) Inventors: Jeong-Chan KIM, Daejeon (KR); Ki-Sung SUNG, Incheon (KR); Gi-Tak CHAE, Yongin-si (KR)

(21) Appl. No.: 14/332,179

(22) Filed: Jul. 15, 2014

(30) Foreign Application Priority Data
Jul. 31, 2013 (KR) 10-2013-0090823

(51) Int. Cl. G01N 33/18; G01N 27/00; G01N 25/00; G01N 21/00

(52) U.S. Cl. CPC G01N 33/18; G01N 21/00; G01N 27/00; G01N 25/00
USPC 73/61.61

(57) ABSTRACT

Disclosed are an apparatus for measuring outer water quality in a remote place, which is fixedly provided at a specific region having a water source so that a position of a water quality sensor is controlled in a remote place based on a real-time moving picture transmitted from a measurement area and the water quality sensor is inserted into water, thereby measuring water quality, and a method thereof. A horizontal position of a sensor holder is determined by adjusting a horizontal length variable rod in a remote place, a sensing part of at least one water quality sensor provided in the sensor holder is inserted into water by adjusting a first vertical length variable rod and a second vertical length variable rod in the remote place, a measurement value measured from the water quality sensor is transmitted to a terminal in the remote place, and the measurement value is displayed.

### CROSS-REFERENCE TO RELATED APPLICATION

This application claims the benefit under 35 U.S.C. §119 of Korean Patent Application No. 10-2013-0090823 filed on Jul. 31, 2013 in the Korean Intellectual Property Office, the entirety of which disclosure is incorporated herein by reference.

### TECHNICAL FIELD

The present invention relates to an apparatus for measuring outer water quality in a remote place, which is fixedly provided at a specific region having a water source so that a position of a water quality sensor is controlled in a remote place based on a real-time moving picture transmitted from a measurement area and the water quality sensor is inserted into water, thereby measuring water quality, and a method thereof.

### BACKGROUND ART

Wastewater discharged from enterprises and household sewage are increased due to high economic growth, so that water quality is significantly deteriorated in rivers or reservoirs. Water is closely related to a human life, and water pollution considerably destructs surrounding ecological environments, which may exert a harmful influence on the human life. Therefore, studies and research on the technology of measuring a water pollution level to remove a water pollution source has been actively performed.

The water pollution is mainly classified into underground pollution, river water pollution, and seawater pollution.

To measure the water pollution level, in the case of underground water, a borehole is excavated under the ground to measure the pollution level of underground water. In the case of the river water or the seawater, a tester personally moves to a measurement area to measure a water quality level.

As the related art of the present invention, there is Korean Unexamined Patent Publication No. 2012-0076517 (published on Jul. 9, 2012). The related art discloses a low-power intelligent water quality monitoring apparatus including a sensor unit to generate water quality data by measuring the water quality, a storage unit to store the water quality data according to the control of a control unit, a communication unit to wirelessly transmit the water quality data to an integrated management center, and the control unit to control the operations of the sensor unit and the communication unit according to the water quality level of the water quality data in such a manner that the sensor unit and the communication unit are turned on for a water quality measurement period and turned off for other times.

However, according to the related art, since a plurality of water quality sensors are provided under ground, and driven only if a pollution level is equal to or greater than a critical level, the water quality of a specific region cannot be measured. If a sensor is left under flowing water for a long time, the endurance of the water quality sensors is sharply decreased, so that the lifespan of the water quality sensors is shortened.

In addition, since the sensor receives power from a power source such as a battery embedded therein for the operation thereof, the sensor may not be used for a long time.

Accordingly, a scheme of remotely measuring water quality based on a real-time moving picture photographed in a water source at a specific region and automatically measuring the water quality for a set time has been strongly required.

### DISCLOSURE

### Technical Problem

The present invention has been made keeping in mind the above problem occurring in the art, and an object of the present invention is to provide an apparatus for remotely measuring outdoor water quality, in which the water quality is remotely measured based on a moving picture transmitted in real time from a specific region, and self-generation is possible so that a long time use is possible without replacement of power.

The present invention has been made keeping in mind the above problem occurring in the art, and an object of the present invention is to provide an apparatus for remotely measuring outdoor water quality, in which the water quality is remotely measured based on a moving picture transmitted in real time from a specific region, and self-generation is possible so that a long time use is possible without replacement of power.

### Technical Solution

In order to accomplish the above object, there is provided an apparatus for remotely measuring outdoor water quality. The apparatus includes a sensor holder having at least one water quality sensor, a camera to photograph the sensor holder in real time, a first vertical length variable rod provided at one end portion thereof with the sensor holder to move the sensor holder up or down, a horizontal length variable rod having one end portion provided at an opposite end portion of the first vertical length variable rod to move the sensor holder back and forth in a horizontal direction, a second vertical length variable rod provided at an opposite end portion of the horizontal length variable rod to move the sensor holder up or down in a vertical direction, a control box that receives commands of controlling lengths of the first vertical length variable rod, the horizontal length variable rod, and the second vertical length variable rod according to a moving picture photographed by the camera from a remote place to insert the sensor holder into water and performs the commands, and a battery that supplies power. A terminal comprising a second communication unit embedded therein to transmit the commands to the control box and to receive a measurement value measured by the water quality sensor and the moving picture photographed in real time from the camera is provided in the remote place.

The apparatus further includes a solar thermal power generator to produce electricity by using solar heat to fill the battery with the electricity, and the solar thermal power generator includes a photo-sensor to fold a photo-receiving area if intensity of received light is equal to or lower than a preset critical value. The apparatus further includes a first motor to move the horizontal length variable rod in a horizontal direction and to move the first vertical length variable rod in a vertical direction, and a second motor to move the second vertical length variable rod in a vertical direction.

In addition, the control box includes a first communication unit to wirelessly receive the commands from the second communication unit and to wirelessly transmit the measurement value measured by the water quality sensor and the moving picture photographed by the camera to the second communication unit, a horizontal length control unit to control a horizontal length of the horizontal length variable rod, a first vertical length control unit to control a vertical length of the first vertical length variable rod, a second vertical length control unit to control a vertical length of the second vertical length variable rod, a storage unit to store information sensed from the water quality sensor, and a central control unit to control the horizontal length control unit, the first vertical length control unit, and the second vertical length control unit according to the commands. The first vertical length control unit more finely controls a length as compared with the second vertical length control unit.

Meanwhile, the apparatus further includes a setting unit to transmit the commands to the central control unit at a preset time if the commands are previously input such that the central control unit performs the control operations at the preset time, and further includes a self-diagnosing unit to detect an abnormal state of the apparatus for remotely measuring the outdoor water quality and to generate an alarm message to be transmitted to the terminal in the remote place.

Further, the terminal includes a stationary terminal and a mobile terminal, and the second communication unit wirelessly transceives information, the second communication unit wirelessly transceives information, and the terminal further includes an input unit to receive the commands, and a display unit to display the commands and the measurement value.

In addition, the apparatus further includes a water quality sensor cover to protect the water quality sensor from an external environment including rain or snow.

Meanwhile, to this end, there is provided a method of remotely measuring outer water quality, which includes (A) determining a horizontal position of a sensor holder by adjusting a horizontal length variable rod in a remote place, (B) inserting a sensing part of at least one water quality sensor provided in the sensor holder into water by adjusting a first vertical length variable rod and a second vertical length variable rod in the remote place, (C) transmitting a measurement value measured from the water quality sensor to a terminal in the remote place, and (D) displaying the measurement value transmitted to the terminal.

In addition, step (D) includes (E) detecting an abnormal state and transmitting an alarm message to the terminal.

In addition, the water quality sensor includes a Ph sensor, an EC sensor, a DO sensor, a temperature (T) sensor, and an ORP sensor.

The advantages and features of the present invention will be apparently comprehended by those skilled in the art based on the following detailed description made with reference to accompanying drawings.

Terms and words used in the specification and the claims shall not be interpreted as commonly-used dictionary meanings, but shall be interpreted as to be relevant to the technical scope of the invention based on the fact that the inventor may properly define the concept of the terms to explain the invention in best ways.

### Advantageous Effects

According to various embodiments of the present invention, the water quality sensor is inserted into water through the remote control in a remote place based on a real time moving picture transmitted by a camera, so that a pollution level can be measured.

According to various embodiments of the present invention, since a solar thermal power generator in which self-power generation is possible is provided, water quality can be measured in an outdoor place without a battery for a long time.

In addition, according to various embodiments of the present invention, since settings to measure water quality at a specific time point are possible, the water quality can be automatically measured for a specific period of time at the specific time point.

### DESCRIPTION OF DRAWINGS

FIG. 1 is a view showing an example of an apparatus for remotely measuring outdoor water quality according to one embodiment of the present invention.

FIG. 2 is a block diagram schematically showing a control box in the apparatus for remotely measuring outdoor water quality according to the embodiment of the present invention.

FIG. 3 is a block diagram schematically showing a terminal in the apparatus for remotely measuring outdoor water quality according to the embodiment of the present invention.

FIG. 4 is a block diagram showing a self-diagnosing unit in the apparatus for remotely measuring outdoor water quality according to the embodiment of the present invention.

FIG. 5 is a view an example of a solar thermal power generator in the apparatus for remotely measuring outdoor water quality according to the embodiment of the present invention.

FIG. 6 is a view showing another example of a solar thermal power generator in the apparatus for remotely measuring outdoor water quality according to the embodiment of the present invention.

FIG. 7 is a flowchart sequentially showing a method of remotely measuring outdoor water quality according to another embodiment of the present invention.

### BEST MODE

### Mode for Invention

The objects, the specific advantages, and the novel features of the present invention will be apparently comprehended by those skilled in the art based on the embodiments, which are detailed later in detail, together with accompanying drawings. In the following description, the same reference numerals will be used to refer to the same elements throughout the drawings. Although the terms “first” and “second” may be used in the description of various elements, the embodiment is not limited thereto. The terms “first” and “second” are used to distinguish one element from the other elements.

As used herein, the singular forms “a”, an and the are intended to include the plural forms as well, unless the context clearly indicates otherwise. In the following description, when a predetermined part “includes” a predetermined component, the predetermined part does not exclude other components, but may further include other components if there is a specific opposite description.

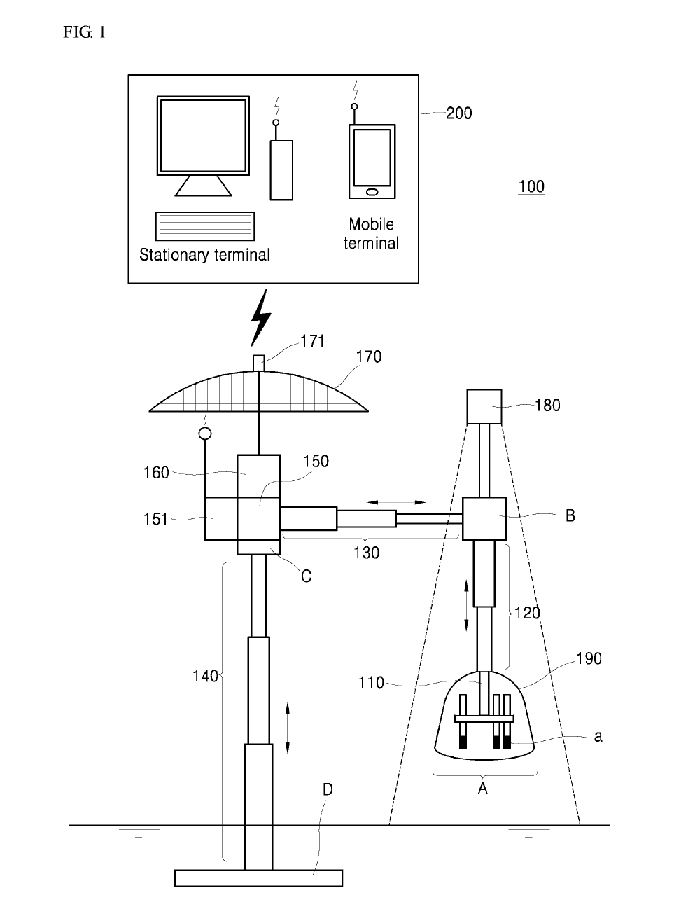

In the following description referring to FIGS. 1 to 7, the same reference numerals will be assigned to the same elements.

The basic principle of the present invention is as follows. An apparatus for remotely measuring outer water quality is fixedly provided at a specific region having a water source and remotely controlled based on a real-time moving picture transmitted from a measurement area to insert a sensor holder into water, so that water quality is measured by a water quality sensor provided in the sensor holder.

In the following description, if detailed description about well-known functions or configurations may make the subject matter of the disclosure unclear, the detailed description will be omitted.

Hereinafter, an exemplary embodiment of the present invention will be described in detail with reference to accompanying drawings.

FIG. 1 is a view showing an example of an apparatus for remotely measuring outdoor water quality according to one embodiment of the present invention.

Referring to FIG. 1, an apparatus 100 for remotely measuring outdoor water quality according to the present invention includes a sensor holder 110 having at least one water quality sensor, a camera 180 to photograph the sensor holder 110 in real time, a first vertical length variable rod 120 provided at one end portion thereof with the sensor holder 110 to move the sensor holder 110 up or down, a horizontal length variable rod 130 having one end portion provided at an opposite end portion of the first vertical length variable rod 120 to move the sensor holder 110 back and forth in a horizontal direction, a second vertical length variable rod 140 provided at an opposite end portion of the horizontal length variable rod 130 to move the sensor holder 110 up or down in a vertical direction, a control box 150 to receive commands of controlling the lengths of the first vertical length variable rod 120, the horizontal length variable rod 130, and the second vertical length variable rod 140 according to the moving picture photographed by the camera 180 in order to insert the sensor holder 110 into water and to perform the commands, and a battery to supply power.

Hereinafter, the apparatus 100 for remotely measuring outdoor water quality according to the embodiment of the present invention configured as shown in FIG. 1 will be described in detail.

First, the apparatus 100 for remotely measuring outdoor water quality according to the present invention is provided at an outdoor area in order to measure the outdoor water quality.

For example, the apparatus 100 for remotely measuring outdoor water quality is fixedly provided in underground water stagnated in a borehole or at a specific area, such a lake, a river, or a beach, having a water source to insert the sensor holder 110 into water according to real-time moving pictures transmitted from a measurement area to measure water quality from a water quality sensor A provided in the sensor holder 110.

Therefore, disclosed is a sensor holder 110 having at least one water quality sensor A.

The water quality sensor A is coupled with the sensor holder 110, and a sensing part a of the water quality sensor A is inserted into water to measure various water quality characteristics.

The water quality sensor A may include a Ph sensor, an EC sensor, a DO sensor, a temperature (T) sensor, or an ORP sensor, but the embodiment is not limited thereto.

In this case, the sensor holder 110 is positioned above the sensing part a of the water quality sensor A. Accordingly, if the sensor holder 110 is inserted into water, the sensing part a is inserted into water to measure water quality.

In addition, since the water quality sensor A, especially, the sensing part a is sensitive, the sensor holder 100 is additionally provided therein with a water quality sensor cover 190 to protect the water quality sensor A from an external environment such as rain or snow.

In this case, the water quality sensor cover 190 may include a transparent material or an opaque material. Preferably, the water quality sensor cover 190 includes a transparent material such as synthetic resin or tempered glass. In addition, the water quality sensor cover 190 may include various materials only if the materials do not exert an influence on the measured value by the water quality sensor A while protecting the water quality sensor A.

Hereinafter, an operation of inserting the sensor holder 110 into water to be measured by adjusting the first vertical length variable rod 120 coupled with a rear end portion of the sensor holder 110, the horizontal length variable rod 130 coupled with a rear end portion of the first vertical length variable rod 120 in a horizontal direction, and the vertical length variable rod 140 coupled with the rear end of the horizontal length variable rod 120 in a vertical direction will be described in detail.

The operation of inserting the sensor holder 110 into water is mainly classified into two operations.

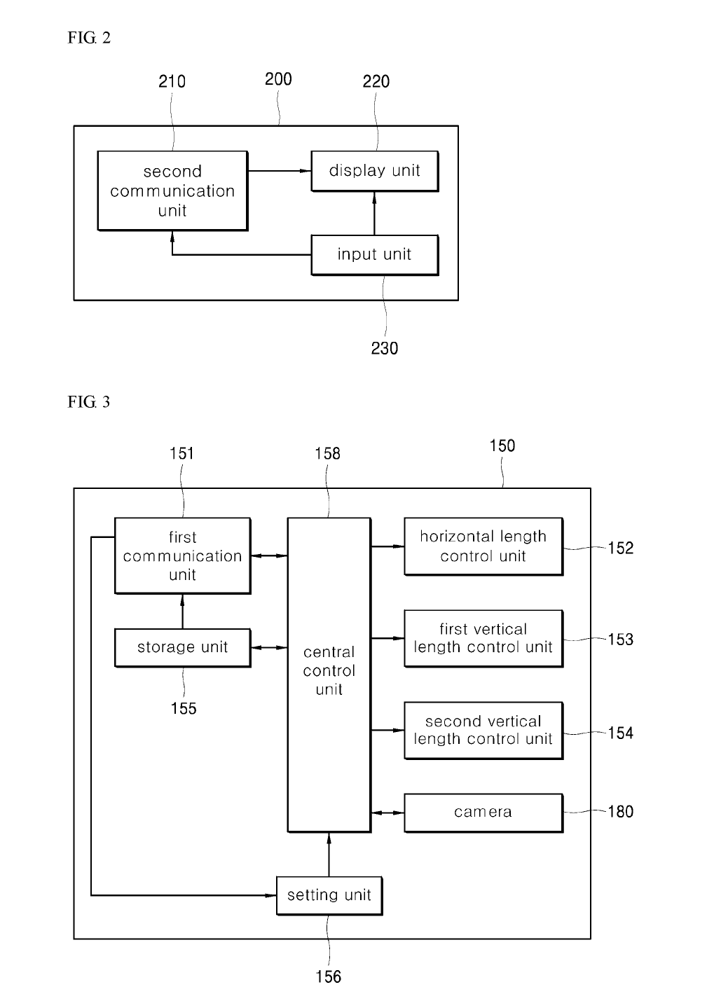

One of the operations is performed by displaying the moving picture photographed from the camera 180 in a remote plate and transmitting the commands of controlling the first vertical length variable rod 120, the horizontal length variable rod 130, and the second vertical length variable rod 140 to the control box 150. The other is performed by previously inputting the commands of controlling the first vertical length variable rod 120, the horizontal length variable rod 130, and the second vertical length variable rod 140 into a setting unit 156 of the control box 150 shown in FIG. 2 and repeatedly performing the commands at a specific time.

First, hereinafter, the former will be described in detail.

In the following detailed description of the former, the former will be described with reference to FIGS. 1 to 3.

FIG. 2 is a block diagram schematically showing the control box 150 in the apparatus 100 for remotely measuring outdoor water quality according to the embodiment of the present invention. FIG. 3 is a block diagram schematically showing a terminal 200 in the apparatus 100 for remotely measuring outdoor water quality according to the embodiment of the present invention.

The camera 180 provided above the sensor holder 110 is powered on or off by the terminal 200 in a remote place and include a small motor (not shown) to finely move in order to change a photographing angle.

In this case, the terminal 200 is classified into a stationary terminal and a mobile terminal. The stationary terminal is mainly referred to as a personal computer, and the mobile terminal is mainly referred to as a smart phone.

If a command of turning on the camera 180 is input from an input unit 230 of the terminal 200, a second communication unit 210 wirelessly transmits the command to a first communication unit 151.

If the first communication unit 151 transmits the command to a central control unit 158, the central control unit 158 turns on the camera 180. Thereafter, the moving picture photographed by the camera 180 is wirelessly transmitted to the second communication unit 210 through the first communication unit 151. Then, the transmitted moving picture is reproduced on the display unit 220, so that the position of the sensor holder 110 may be checked.

In order to insert the sensing part a of the water quality sensor A into water according to the checked position of the sensor holder 110, the first vertical length variable rod, the horizontal length variable rod, and the second vertical length variable rod 120, 130, and 140 must be controlled.

To this end, if the command of controlling the first vertical length variable rod, the horizontal length variable rod, and the second vertical length variable rod 120, 130, and 140 is input through the input unit 230, the command is displayed on the display unit 220 so that the command is primarily checked, and wirelessly transmitted to the first communication unit 151 through the second communication unit 210.

The first communication unit 151 transmits the command of controlling the first vertical length variable rod 120, the horizontal length variable rod 130, and the second vertical length variable rod 140 to respective control units to control the first vertical length variable rod 120, the horizontal length variable rod 130, and the second vertical length variable rod 140.

For example, if the command of controlling the first vertical length variable rod 120 is received, the command is transmitted to a first vertical length control unit 153. If the command of controlling the second vertical length variable rod 140 is received, the command is transmitted to a second vertical length control unit 154. If the command of controlling the horizontal length control unit 152 is received, the command is transmitted to a horizontal length control unit 152.

In this case, the camera 180 photographs the sensor holder 110, the position of which is changed, in real time and transmits the moving picture of the sensor holder 110 to the mobile terminal 200.

Thereafter, if the sensing part a of the water quality sensor A coupled with the sensor holder 110 is inserted into water, the commands of moving the first vertical length variable rod 120, the horizontal length variable rod 130, and the second vertical length variable rod 140 are stopped.

In this case, both of the first vertical length variable rod 120 and the second vertical length variable rod 140 may be used to adjust the vertical position of the sensor holder 110. However, the vertical length can be adjusted to a longer length through the second vertical length variable rod 140 rather than the first vertical length variable rod 120, and can be more finely adjusted through the second vertical length variable rod 140 rather than the first vertical length variable rod 120.

Finally, after the measured value by the water quality sensor A has been stored in a storage unit 155 by the central control unit 158, the measured value is wirelessly transmitted to the mobile terminal 200 and displayed.

In this case, a measurement time, and environment information of the temperature and the moisture at a measurement area are stored in the storage unit 155 together with the measured value. Accordingly, preferably, the control box 150 includes a clock, a thermometer, or a hydrometer.

In addition, after all measuring works have been finished, the mobile terminal 200 wirelessly transmits the commands of controlling the first vertical length variable rod 120, the horizontal length variable rod 130, and the second vertical length variable rod 140, so that the sensor holder 110 is moved out of water.

In this case, for the continuous measuring works, the sensing part a may be left in water till required time.

Hereinafter, the latter, that is, the operation of previously inputting the commands of controlling the first vertical length variable rod 120, the horizontal length variable rod 130, and the second vertical length variable rod 140 into the setting unit 156 of the control box 150 and repeatedly performing the commands at a specific time will be described in detail.

First, the mobile terminal 200 wirelessly receives the commands for controlling the first vertical length variable rod 120, the horizontal length variable rod 130, and the second vertical length variable rod 140.

In this case, the insertion distance of the sensing part a inserted into water is known.

For example, the sensor holder 110 is positioned at a default region of the apparatus 100 for remotely measuring outdoor water quality. In other words, if the apparatus 100 for remotely measuring outdoor water quality is reset, the sensor holder 110 is positioned at a default region. Therefore, the moving distances of the sensing part a in horizontal and vertical direction from the default region to insert the sensing part a into water can be recognized.

The mobile terminal 200 inputs horizontal and vertical distances of the sensing part a to be moved from the default region, a time to insert the sensing part a into water, and a measurement time by the sensing part a to the first communication unit 151 in order to insert the sensing part a into water and wireless transmits the horizontal and vertical distances of the sensing part a, the time to insert the sensing part a into the water, and the measurement time by the sensing part a to the first communication unit 151.

Then, the central control unit 158 may transmit control commands to the horizontal length control unit 152, the first vertical length control unit 153, the second vertical length control unit 154 at a preset time point to operate the first vertical length variable rod 120, the horizontal length variable rod 130, and the second vertical length variable rod 140 so that the sensing part a is inserted into water during a preset time to measure the water quality.

If the preset time is elapsed, the position of the sensor holder 110 is reset and the sensor holder 110 is moved to the default region.

Meanwhile, the first vertical length variable rod 120 and the horizontal length variable rod 130 are operated by a first motor B, and the second vertical length variable rod 140 is operated by a second motor C. In addition, a support plate D is provided at a lower end portion of the second vertical length variable rod 140, so that the apparatus 100 for remotely measuring outdoor water quality can be stably erected.

Although a unit of erecting the apparatus 100 for remotely measuring outdoor water quality is employed as the support plate D in the description of the present invention, a lower portion of the second vertical length variable rod 140 is buried underground, so that the second vertical length variable rod 140 can be stably erected.

Referring to FIG. 1 again, the apparatus 100 for remotely measuring outdoor water quality includes a battery 160. The battery 160 consumes power while serving as a power source to supply power to the apparatus 100 for remotely measuring outdoor water quality.

If a preset time is elapsed, the battery 160 must be replaced with new one or recharged. Accordingly, a solar thermal power generator 170 is provided above the battery 160, so that the battery 160 may be charged with power.

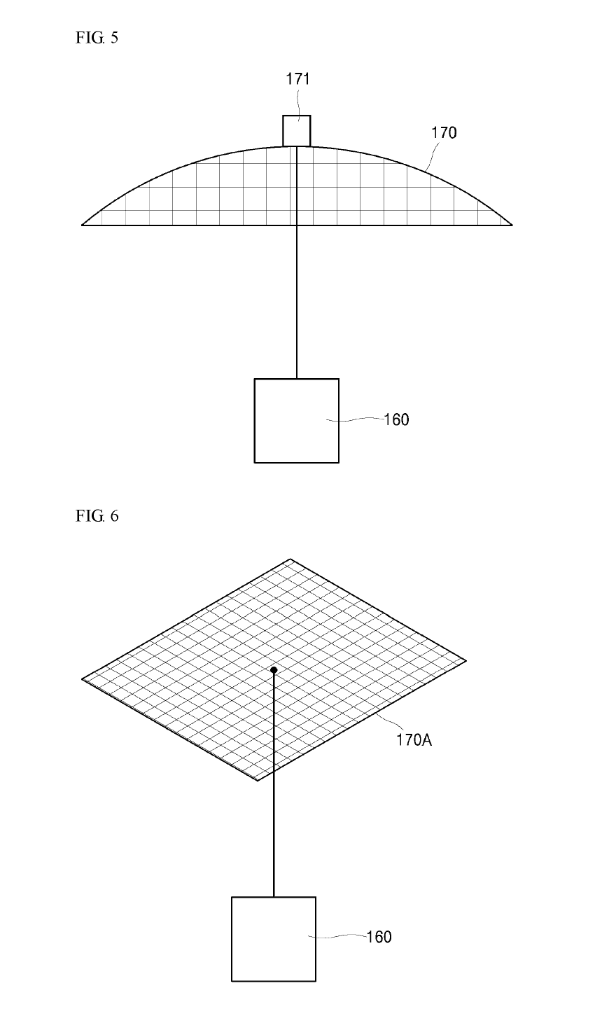

The solar thermal power generator 170 will be described later with respect to FIGS. 5 and 6.

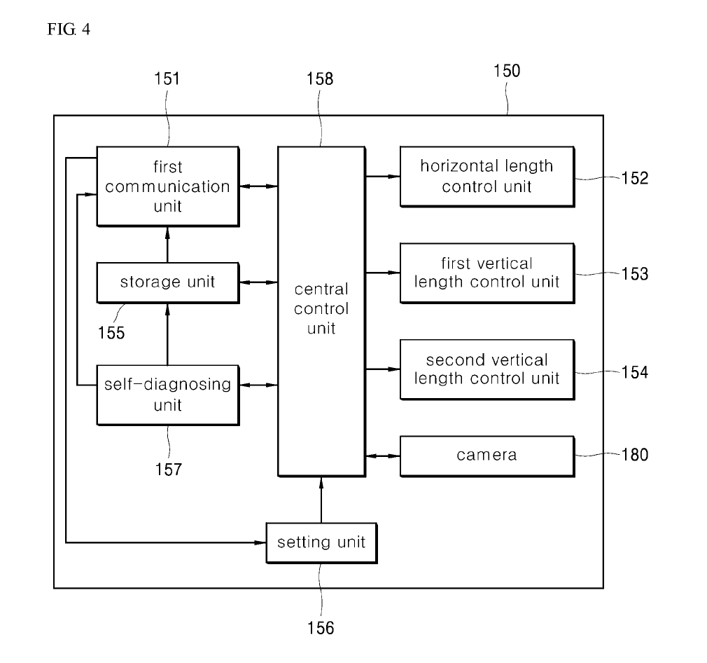

Referring to FIG. 4 again, a self-diagnosing unit 157 according to one embodiment of the present invention will be described in detail.

FIG. 4 is a block diagram showing the self-diagnosing unit 157 in the apparatus 100 for remotely measuring outdoor water quality according to the embodiment of the present invention.

In the following description referring to FIG. 4, the structure and the components the same as those of FIG. 1 will not be further described.

The reason why the self-diagnosing unit 157 is provided in the control box 150 according to the embodiment of the present invention is as follows. The components of the apparatus 100 for remotely measuring outdoor water quality under an outdoor environment have high failure possibility. Accordingly, the self-diagnosing unit 157 detects the failure of the component and transmits an alarm message related to the failure of the component to the mobile terminal 200 in failure, so that the apparatus for remotely measuring outdoor water quality can easily cope with the failure.

In particular, the self-diagnosing unit 157 is electrically connected with the first motor B, the second motor C, the battery 160, and the camera 180 to detect the erroneous operations of the first motor B, the second motor C, the battery 160, and the camera 180 if the erroneous operations of the first motor B, the second motor C, the battery 160, and the camera 180 occur. Then, the self-diagnosing unit 157 provides information of the erroneous device and an alarm message to the mobile terminal 200.

Therefore, the mobile terminal 200 receives and displays the information of the erroneous device and the alarm message.

FIG. 5 is a view an example of a solar thermal power generator in the apparatus for remotely measuring outdoor water quality according to the embodiment of the present invention.

Referring to FIG. 5, the solar thermal power generator 170 according to the embodiment of the present invention may have the shape of an umbrella that is foldable.

A photo-sensor 171 is provided at the most upper part of photoelectric devices provided in a lattice shape. If the photo-sensor 171 detects luminous intensity lower than a critical value in which solar thermal power cannot be generated, the photo-sensor 171 provides this information to the central control unit 158. Accordingly, the central control unit 158 folds light receiving areas of the photoelectric devices of the solar thermal power generator 170 arranged in the lattice shape

In this case, if the photo-sensor 171 detects the luminous intensity equal to or greater than the critical value in which the solar thermal power generation is possible, the photo-sensor 171 provides the information that the luminous intensity is equal to or greater than the critical value to the central control unit 158. Accordingly, the central control unit 158 spreads a photo detecting area of the solar thermal power generator 170, so that the solar thermal power generation is performed.

Therefore, the power generated from the solar thermal power generator 170 is supplied to the battery 160 to compensate for power consumption of the battery 160, so that the lifespan of the battery 160 can be improved.

As described above, the folding and the spreading of a photo-detecting area are controlled, thereby preventing a photo-detector from being erroneously operated as the photo-detector is exposed to rain, snow, or an external environment for a long time.

FIG. 6 is a view showing another example of a solar thermal power generator 170A in the apparatus 100 for remotely measuring outdoor water quality according to the embodiment of the present invention.

Referring to FIG. 6, a solar thermal power generator 170A according to the embodiment of the present invention has the shape of a flat plate and is always provided above the battery 160. The power generated from the solar thermal power generator 170A is supplied to the battery 160 and compensates for the consumed power of the battery 160.

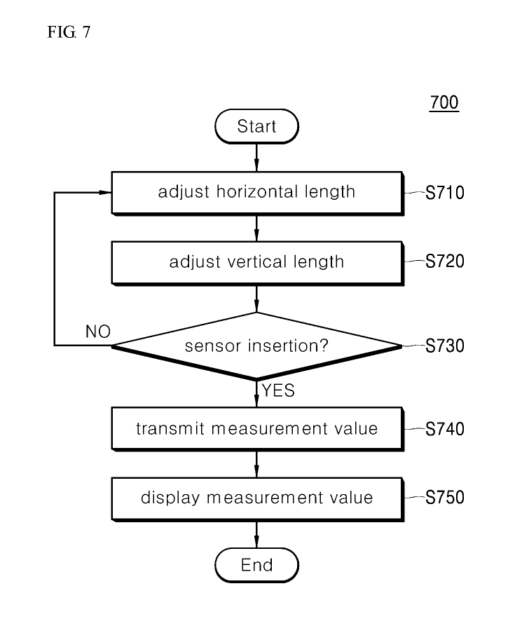

FIG. 7 is a flowchart sequentially showing a method 700 of measuring outdoor water quality according to another embodiment of the present invention.

Referring to FIG. 7, the method 700 of measuring the outdoor water quality according to another embodiment of the present invention includes a step (S710) of determining a horizontal position of a sensor holder by adjusting the horizontal length variable rod 130 in a remote place (step S710); a step (S720) of inserting the sensing part a of at least one water quality sensor A provided in the sensor holder 110 into water by adjusting the first vertical length variable rod 120 and the second vertical length variable rod 140 in the remote place; a step (S730) of transmitting a measurement value measured from the water quality sensor A to the terminal 200 in the remote place; and a step (S740) of displaying the measurement value transmitted to the terminal 200.

Hereinafter, the method 700 of measuring the outdoor water quality according to another embodiment of the present invention having process steps shown in FIG. 7 will be described.

First, the horizontal length variable rod 130 is adjusted in the remote place to determine the horizontal position of the sensor holder 110 (step S710).

The horizontal length variable rod 130 has one end portion connected with an opposite end portion of the first vertical length variable rod 120, and the first vertical length variable rod 120 has one end portion connected with the sensor holder 110.

Accordingly, the horizontal position of the sensor holder 110 is determined by the horizontal length variable rod 130.

In this case, at least one water quality sensor A is coupled with the sensor holder 110, and the sensing part a is provided at a lower portion of the water quality sensor A to measure water quality. The sensing part a is inserted into water to measure the state of the water quality.

The water quality sensor A may include a Ph sensor, an EC sensor, a DO sensor, a temperature (T) sensor, and an ORP sensor, but the embodiment is not limited thereto.

Thereafter, the first vertical length variable rod 120 and the second vertical length variable rod 140 are adjusted in the remote place so that the sensing part a of at least one water quality sensor A provided in the sensor holder 110 is inserted into water (step S720).

All of the first vertical length variable rod 120 and the second vertical length variable rod 140 can control the vertical position of the sensor holder 110. Particularly, the second vertical length variable rod 140 can control a vertical distance longer than that of the first vertical length variable rod 120, and the first vertical length variable rod 120 can more finely the vertical distance as compared with the second vertical length variable rod 140.

The measurement value measured by the water quality sensor A is wirelessly transmitted to the terminal 200 in the remote place (step S730), and the terminal 200 displays the transmitted measurement value on the display unit 220 (step S740).

The measurement value is displayed as described above, so that the information of the water quality in a remote place can be easily recognized.

As described above, although various examples have been illustrated and described, the present disclosure is not limited to the above-mentioned examples and various modifications can be made by those skilled in the art without departing from the scope of the appended claims. In addition, these modified examples should not be appreciated separately from technical spirits or prospects.

1. An apparatus for remotely measuring outdoor water quality, the apparatus comprising:
a sensor holder having at least one water quality sensor;
a camera to photograph the sensor holder in real time;
a first vertical length variable rod provided at one end portion thereof with the sensor holder to move the sensor holder up or down;
a horizontal length variable rod having one end portion provided at an opposite end portion of the first vertical length variable rod to move the sensor holder back and forth in a horizontal direction;
a second vertical length variable rod provided at an opposite end portion of the horizontal length variable rod to move the sensor holder up or down in a vertical direction;
a control box that receives commands of controlling lengths of the first vertical length variable rod, the horizontal length variable rod, and the second vertical length variable rod according to a moving picture photographed by the camera from a remote place to insert the sensor holder into water and performs the commands; and
a battery that supplies power,
wherein a terminal comprising a second communication unit embedded therein to transmit the commands to the control box and to receive a measurement value measured by the water quality sensor and the moving picture photographed in real time from the camera is provided in the remote place.

2. The apparatus of claim 1, further comprising a solar thermal power generator to produce electricity by using solar heat to fill the battery with the electricity, wherein the solar thermal power generator comprises a photo-sensor to fold a photo-receiving area if intensity of received light is equal to or lower than a preset critical value.

3. The apparatus of claim 1, further comprising a first motor to move the horizontal length variable rod in a horizontal direction and to move the first vertical length variable rod in a vertical direction; and
a second motor to move the second vertical length variable rod in a vertical direction.

4. The apparatus of claim 1, wherein the control box comprises:
a first communication unit to wirelessly receive the commands from the second communication unit and to wirelessly transmit the measurement value measured by the water quality sensor and the moving picture photographed by the camera to the second communication unit;
a horizontal length control unit to control a horizontal length of the horizontal length variable rod;
a first vertical length control unit to control a vertical length of the first vertical length variable rod;
a second vertical length control unit to control a vertical length of the second vertical length variable rod;
a storage unit to store information sensed from the water quality sensor; and
a central control unit to control the horizontal length control unit, the first vertical length control unit, and the second vertical length control unit according to the commands,
wherein the first vertical length control unit more finely controls a length as compared with the second vertical length control unit.

5. The apparatus of claim 4, further comprising a setting unit to transmit the commands to the central control unit at a preset time if the commands are previously input such that the central control unit performs the control operations at the preset time.

6. The apparatus of claim 1, further comprising a self-diagnosing unit to detect an abnormal state of the apparatus for remotely measuring the outdoor water quality and to generate an alarm message to be transmitted to the terminal in the remote place.

7. The apparatus of claim 1, wherein the terminal comprises a stationary terminal and a mobile terminal, and the second communication unit wirelessly transceives information.

8. The apparatus of claim 1, further comprising a water quality sensor cover to protect the water quality sensor from an external environment including rain or snow.

9. The apparatus of claim 1, wherein the terminal further comprises:
an input unit to receive the commands; and
a display unit to display the commands and the measurement value.

10. A method of remotely measuring outer water quality, the method comprising:
(A) determining a horizontal position of a sensor holder by adjusting a horizontal length variable rod in a remote place;
(B) inserting a sensing part of at least one water quality sensor provided in the sensor holder into water by adjusting a first vertical length variable rod and a second vertical length variable rod in the remote place;
(C) transmitting a measurement value measured from the water quality sensor to a terminal in the remote place; and
(D) displaying the measurement value transmitted to the terminal.

11. The method of claim 10, wherein step (D) comprises (E) detecting an abnormal state and transmitting an alarm message to the terminal.

12. The method of claim 10, wherein the water quality sensor comprises a Ph sensor, an EC sensor, a DO sensor, a temperature (T) sensor, and an ORP sensor.
